(19) United States
(12) Patent Application Publication
(10) Pub. No.: US 2011/0014717 A1
(43) Pub. Date: Jan. 20, 2011

(54) Sensor System and Methods for Chemical Detection

(76) Inventors: Carl A. Marrese, Fayetteville, PA (US); Harry A. Eaton, Columbia, MD (US)

Correspondence Address: THE JOHNS HOPKINS UNIVERSITYAPPLIED PHYSICS LABORA;OFFICE OF PATENT COUNSEL, 11100 JOHNS HOPKINS ROAD, MAIL STOP 7-156, LAUREL, MD, 20723-6099, US

(21) Appl. No.: 12/839,579

(22) Filed: Jul. 20, 2010

Related U.S. Application Data
(60) Provisional application No. 61/226,944, filed on Jul. 20, 2009.

(51) Int. Cl. G01N 33/00; G01N 27/00

(52) U.S. Cl. 436/120; 422/82.01; 436/126; 436/149

(57) ABSTRACT

A miniature system encapsulating a chemical transducer (sensor) prepared for a specific chemical, coupled with a miniature radio transmitter that relays information to a handheld receiver on the presence, or absence, of the target chemical. The system is deployed by several means, such as grenade, small rocket, large caliber rifle, drone plane, etc. The deployment device can hold many such sensor devices, each prepared for a particular toxic chemical, such as chlorine, hydrogen sulfide, or phosgene.

### CROSS-REFERENCE TO RELATED APPLICATIONS

This application relates to U.S. provisional application No. 61/226,944, filed on Jul. 20, 2009, which is incorporated herein by reference in its entirety.

### BACKGROUND OF THE INVENTION

1. Field of the Invention

The present invention relates to a sensor system and methods for remotely detecting chemicals and, more specifically, to a miniature system encapsulating a chemical transducer (sensor) prepared for a specific chemical, coupled with a miniature radio transmitter that relays information to a handheld receiver on the presence, or absence, of the target chemical.

2. Description of the Related Art

Present chemical sensor technology relies on a chemical change to effect a change in a physical, chemical, or electrical property that can be measured. Repeated measurements necessarily imply that the chemical change is reversible, and, therefore, ready to be used again. In practice many chemical sensors employ materials that show reasonable reuse cycles, however, such materials do not show the desired specificity for the target chemical, in other words, most chemical sensors have interferences, that is, chemicals that can cause a false positive.

One chemical of interest to sensor developers is phosgene which is a toxic gas that causes suffocation when inhaled by reacting with proteins in the pulmonary alveoli disrupting the blood air barrier. Levels of phosgene that can be detected by the human olfactory system have been published by several sources, and, consequently, some values vary. Notwithstanding, the NAS/NRC Board on Environmental Studies and Toxicology lists odor perception thresholds for phosgene lying between 0.5 ppm and 1.5 ppm. However, the time-weighted-average (TWA) health threshold limit for phosgene is 0.1 ppm. Consequently, the need to detect phosgene at sub ppm levels is necessary for the continued protection of human life.

Analytical techniques for the detection and quantification of phosgene have been studied for over sixty years. Most methods of phosgene detection rely on the propensity of phosgene to react with many nucleophiles, such as pyridine compounds to form a dye (following subsequent base activation), or quantified by gas chromatographic mass spectrometry. Several commercially available sensor systems are available that employ the common Dräger® tubes, and electrochemical sensors; however, these approaches are either subject to visual observation of a color change, or are plagued with the lack of selectivity. Sensor systems that incorporate selectivity and an automated alarm system are needed.

Noweir and Pfizer developed a colorimetric method for the determination of phosgene using Benzylaniline and 4-nitrobenzylpyridine as their colorimetric reagents; their data showed the detection of phosgene could be made ≦0.1 ppm (Noweir and E. A. Pfitzer, Am. Ind. Hyg. Assoc. J., 1971, 32, 163). Pfizer's approach was modified and studied by Nakano, Yamamoto, Kobayashi, and Nagashima, who developed a colorimetric tape for the detection of phosgene between the concentrations of 0.03 ppm to 0.2 ppm using reflectance spectrophotometry (N. Nakano, A. Yamamoto, Y. Kobayashi, and K. Nagashima, Talanta, 1995, 42, 641). Both investigators successfully employed spectrophotometers to acquire data relating phosgene concentration to a colorimetric signal, however, these approaches were far from handheld sensors or dose meters.

In the military operational theater, soldiers must know if a chemical threat is present, and, as noted above, sensors that have interferences can give false positives. What is needed then is a one time, single use sensor system that is easy to deploy, provides rapid information acquisition, and has specificity for the target chemical thereby eliminating false positives.

### SUMMARY OF THE INVENTION

Therefore, the present invention has been made in view of the above problems and constraints, and provides an apparatus and methods to achieve the above objectives.

More specifically, the present invention is directed to a miniaturized system (for example the size of a marble) encapsulating a chemical transducer (sensor) prepared for a specific chemical, coupled with a miniature radio transmitter that relays information to a handheld receiver on the presence, or absence, of the target chemical. The system can be deployed by many means, such as grenade, small rocket, large caliber rifle, drone aircraft, etc. The deployment device can hold many such sensor systems, each prepared for a particular chemical. Thus, specific sensors can be prepared for toxic chemicals, such as chlorine, hydrogen sulfide, phosgene, etc.

The present invention is further directed to a method for detecting a chemical comprising: coating a light sensor with a formulation that reacts with the chemical to be detected to form a dye, wherein the formulation is transparent to light and the dye, when formed, is opaque; transmitting light to the light sensor; detecting a decrease in voltage of the light sensor when the light sensor is exposed to the chemical causing the dye to form thereby decreasing the amount of transmitted light received by the light sensor; and determining the concentration of the chemical based on the decrease in voltage of the light sensor, the voltage decrease being proportional to the chemical concentration.

The present invention is further directed to a sensor system for detecting a chemical comprising: means for transmitting light; means for sensing the transmitted light; a formulation for coating the sensing means, the formulation comprising a dye forming chemistry, the dye forming chemistry reacting with the chemical to form a dye, the dye being opaque and thereby reducing the light sensed by the sensing means causing a decrease in voltage of the sensing means that is proportional to the concentration of the chemical, the voltage decrease being used to detect the presence of the chemical.

Those and other objects and advantages of the present invention will be fully apparent from the following description, when taken in connection with the annexed drawings.

### BRIEF DESCRIPTION OF THE DRAWINGS

The teachings of the present invention can be readily understood by considering the following detailed description in conjunction with the accompanying drawings, in which:

FIG. 1 illustrates an embodiment of the sensor system of the invention.

FIG. 2 illustrates a second embodiment of the invention for detecting hydrogen sulfide.

FIG. 3, consisting of FIGS. 3A and 3B, illustrates, respectively, coated phototransistors (3) in a printed wiring board apparatus and an uncoated phototransistor compared to a US dime.

FIG. 4 is a diagram of the sensor testing circuitry.

FIG. 5 is a diagram of the sensor test chamber.

FIG. 6 illustrates a UV-Vis spectrum of the phosgene generated dye on a microscope glass slide.

FIG. 7 is a plot of the voltage response from coated glass slides exposed to various concentrations of phosgene in air.

FIG. 8 is a graph of sensor response from a coated phototransistor to 0.244 ppm of phosgene.

FIG. 9 illustrates the electronic circuitry of the radio transmitter of the phototransistor sensor of the invention.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail.

Reference will now be made in detail to the exemplary embodiments of the present invention, examples of which are illustrated in the accompanying drawings.

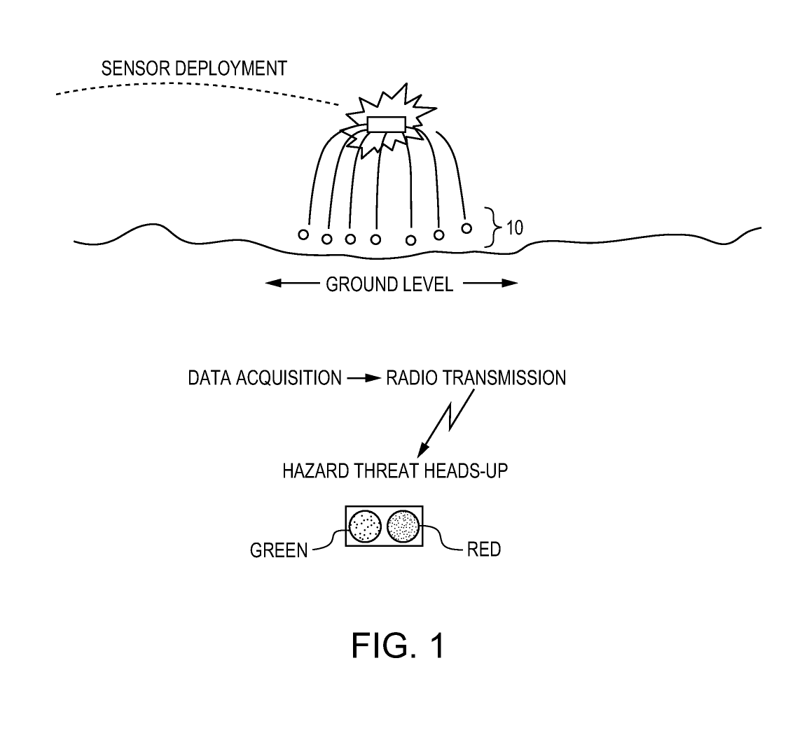

As shown in FIG. 1, a miniature sensor system 10 (for example, the size of a marble) encapsulating a chemical transducer (sensor) prepared for a specific chemical, coupled with a miniature radio transmitter that relays information to a handheld receiver on the presence, or absence, of the target chemical. The device is deployed by several means, such as grenade, small rocket, large caliber rifle, drone plane, etc. The deployment device can hold many such sensor devices, each prepared for a particular chemical. Thus specific sensors can be prepared for toxic chemicals, such as chlorine, hydrogen sulfide, phosgene, etc.

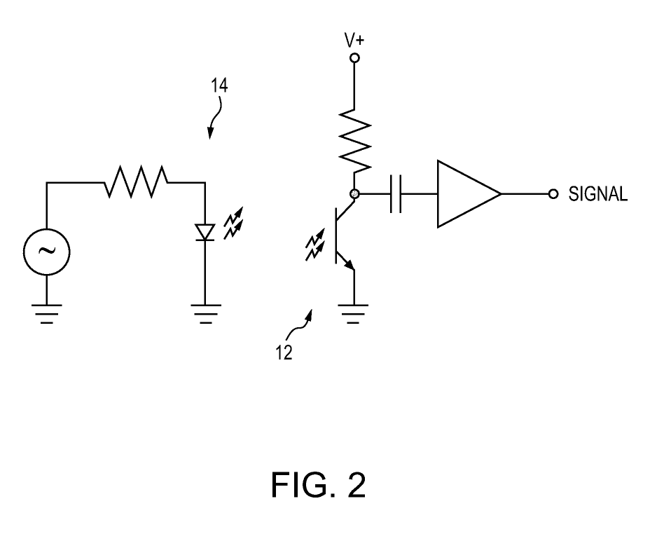

Hydrogen sulfide reacts with lead acetate to form lead sulfide, a dark grayish/black solid. Lead acetate can be deposited on a small light sensor, for example, directly on a phototransistor window 12, as shown in FIG. 2. An LED light source 14 is modulated to eliminate interference from stray light. The sensor will detect hydrogen sulfide as a dimunition of light and relay information to the receiver when the hydrogen sulfide is above a threshold concentration or not, that is, if the signal amplitude is below a threshold level.

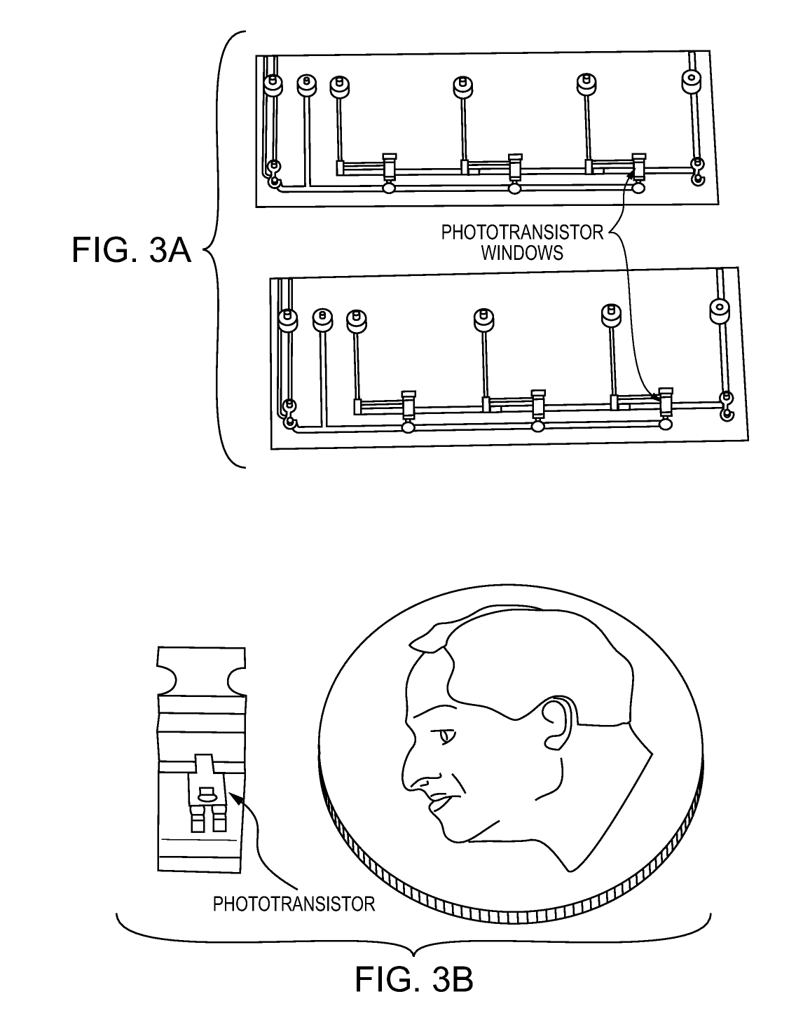

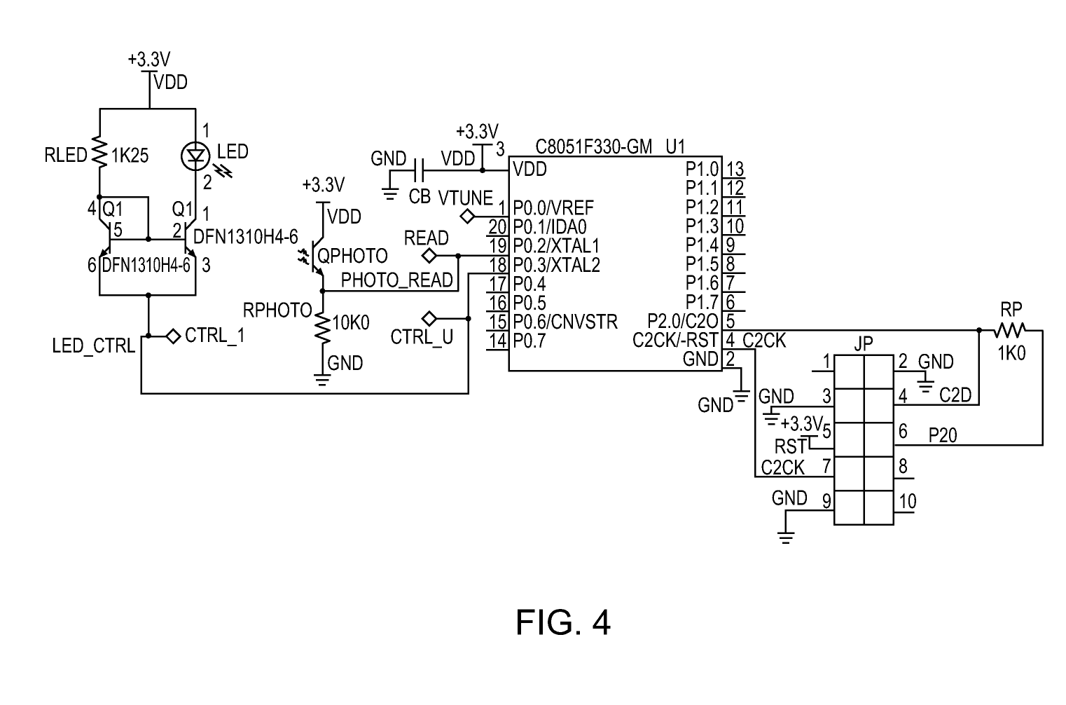

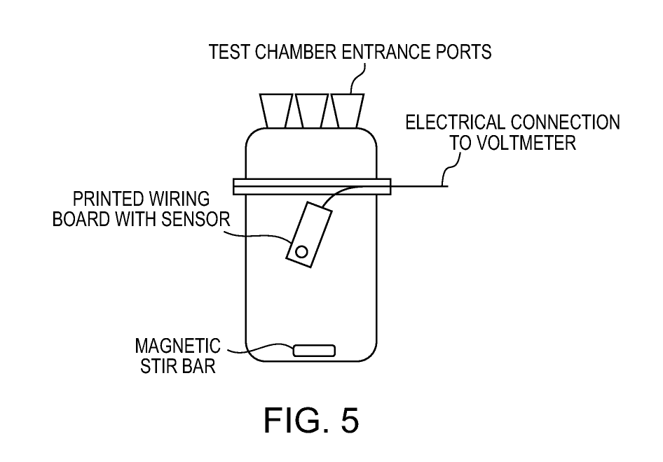

Like hydrogen sulfide, phosgene can also react with a formulation to form a dye in the present invention. Such a formulation can comprise a prepared dichloromethane solution (coating solution) containing the dye forming chemistry consisted of 7.4% 4-(p-Nitrobenzyl)pyridine (4 NP), 14.7% Benzylaniline (BA), and 13.0% Polystyrene (PS) (percentages were based on weight). All chemicals were purchased from Aldrich and were reagent grade or better. Approximately 2 μL of coating solution were applied to microscope glass slides, the same solution and volume was used to coat phototransistor windows, FIG. 3A. (FIG. 3B shows a phototransistor compared to a U.S. dime.) The phototransistors were purchased from Osram Opto Semiconductors, and the LED from Dialight. The LED emission wavelength was 473 nm. Data from coated glass slides and coated phototransistor windows were acquired by the electronic circuitry shown in FIG. 4. A glass reaction vessel, FIG. 5, served as the testing vessel that had a volume of 1.640 L. Phosgene was introduced to the testing vessel by microliter syringe. Various concentrations of phosgene were made by dilution of a 20% (v/v) solution of phosgene in Toluene (Aldrich). Data acquisition was provided by a National Instruments USB data acquisition unit. Glacial acetic acid, and sodium hydroxide pellets were purchased from Fisher Scientific.

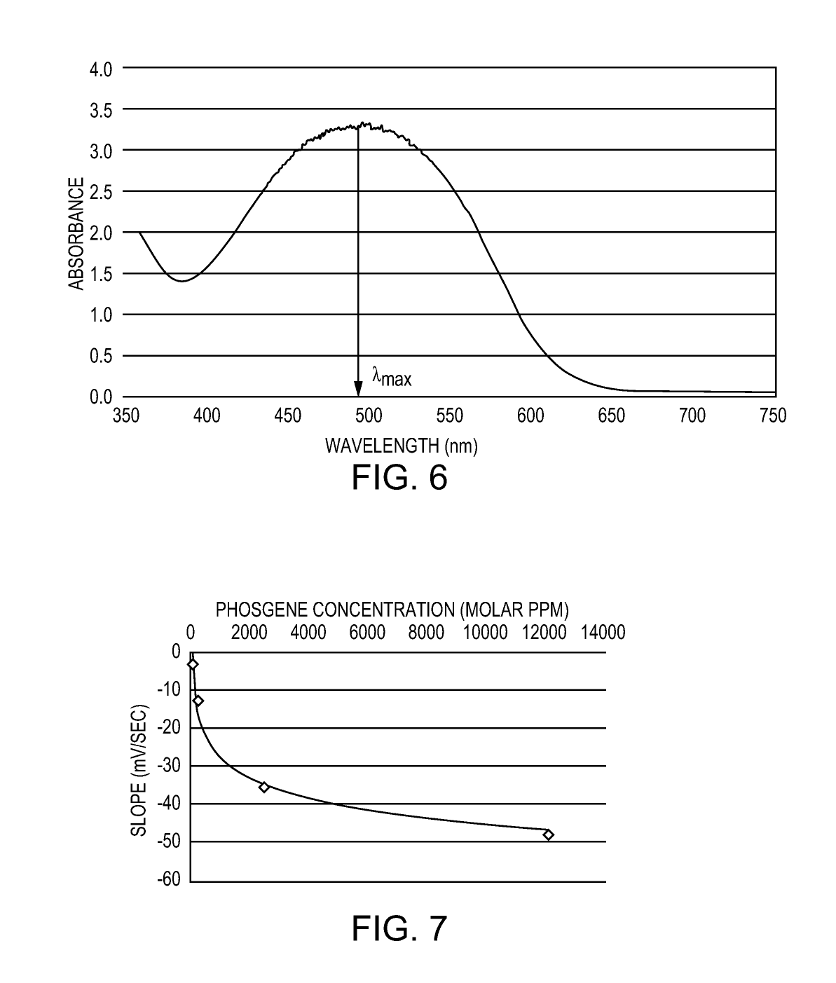

A glass slide was coated with the dye forming chemistry outlined in the experimental section above. The glass slide was placed in the reaction vessel, to which 100 μL, of the 20% phosgene in toluene was added. The coating on the slide turned deep red immediately. FIG. 6 shows the UV-Vis absorption spectrum of the resulting dye formed on the glass slide. This spectrum shows a maximum at approximately 500 nm. Thus, for the phototransistor/LED system, an LED with an emission λₘₐₓ close to the absorption μₘₐₓ of the dye was chosen. Coated glass slides were placed in the slide apparatus shown in FIG. 5, and exposed to various concentrations of phosgene. As dye formed, the voltage of the phototransistor dropped due to dye density obscuring LED light transmission to the phototransistor window.

The slopes of the curves from the onset of voltage change are given in Table 1.

TABLE 1

| Concentration | Slope |
|---|---|
| (ppm) | (mV/sec) |
| 24.4 | −3.096 |
| 244 | −12.758 |
| 2435 | −35.61 |
| 12060 | −48.148 |

A plot of the slopes in Table 1 versus phosgene concentration in the test chamber is shown in FIG. 7. The resulting curve is exponential, and is typical for responses from a surface process, i.e., saturation of the surface.

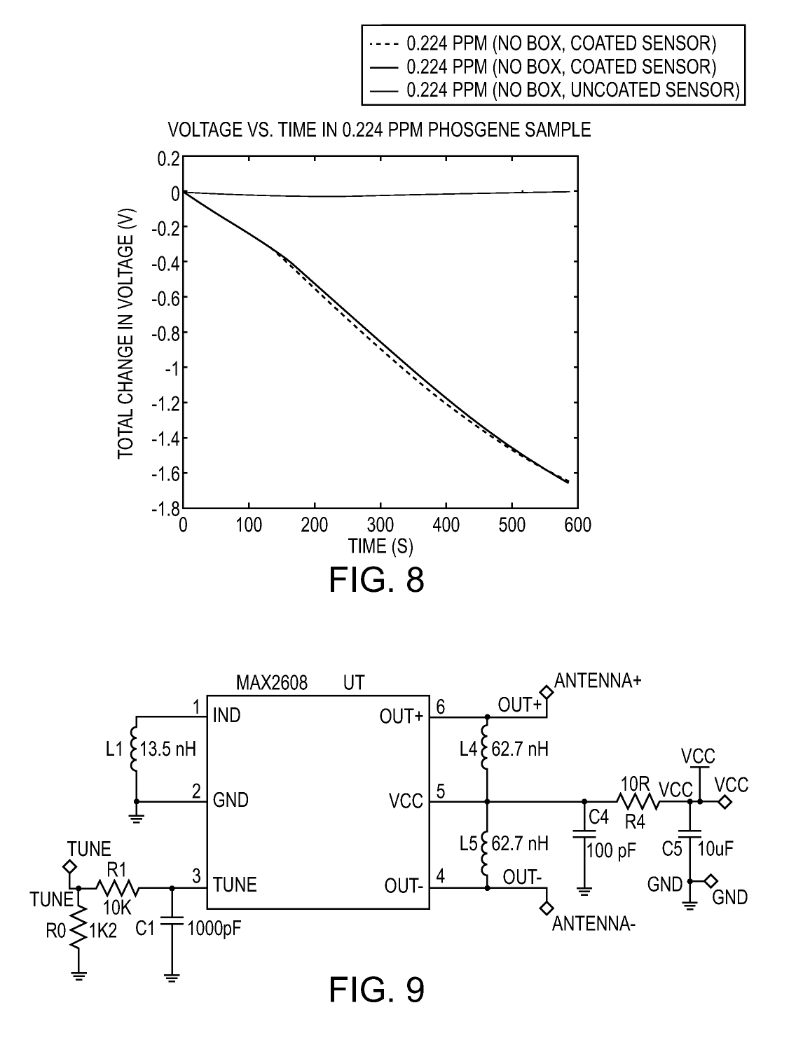

The testing apparatus employed was not an ideal apparatus for determining response time. Consequently, some sensor response curves may not reflect real life responses to phosgene. Nonetheless, a low concentration of phosgene was tested to see the response of the sensor system to sub ppm levels of phosgene. FIG. 8 shows the sensor response to 244 parts per billion (ppb, or 0.244 ppm). The sensor responds in seconds from the time of phosgene introduction. It should be noted that these sensor responses have not been amplified, and represent the raw voltage change that is the direct response of the diminution of light transmission through the phototransistor window due to dye formation.

Ultimately, the sensor system response will be transmitted to a receiver, thus allowing remote sensing of phosgene. FIG. 9 illustrates the electronic circuitry for the sensor system transmitter. The transmitter was tested and at a distance of approximately 750 ft, the synthetic signal (indicting the presence of phosgene over a threshold level) was read by a handheld receiver.

The chemical dye formation is sensitive to phosgene in ambient air, and can be used to form a sensor based on dye density in an LED/phototransistor apparatus. The voltage change from the diminution of light due to dye formation can be used to trigger an alarm preset for a particular phosgene concentration.

The data reported above have demonstrated the feasibility of the invention. It has been shown that this very simple approach of developing a chemical sensor based on selective chemical reaction to yield a dye can yield a response in proportion to the target chemical's airborne concentration.

In addition, as noted, no amplification was made to the electronic circuitry used in the experiment above. A simple modification to the electronic circuitry to amplify the signal will also improve detectability.

It should be apparent to those skilled in the art that the present invention may be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

What is claimed is:

1. A method for detecting a chemical comprising:
coating a light sensor with a formulation that reacts with the chemical to be detected to form a dye, wherein the formulation is transparent to light and the dye, when formed, is opaque;
transmitting light to the light sensor;
detecting a decrease in voltage of the light sensor when the light sensor is exposed to the chemical causing the dye to form thereby decreasing the amount of transmitted light received by the light sensor; and
determining the concentration of the chemical based on the decrease in voltage of the light sensor, the voltage decrease being proportional to the chemical concentration.

2. The method of claim 1, further comprising transmitting the decrease in voltage to a remote receiver.

3. The method of claim 1, further comprising triggering an alarm when the concentration of the chemical reaches a threshold level.

4. The method of claim 1, wherein the light sensor comprises a phototransistor.

5. The method of claim 1, wherein the light is transmitted by an LED.

6. The method of claim 1 wherein the formulation comprises lead acetate and the chemical comprises hydrogen sulfide.

7. The method of claim 1, wherein the formulation comprises one of a dichloromethane, toluene and chloroform solution containing a dye forming chemistry and the chemical comprises phosgene.

8. The method of claim 7, the dye forming chemistry comprising 4-Nitrobenzylpyridine and benzylaniline.

9. The method of claim 8, the dye forming chemistry further comprising polystyrene.

10. The method of claim 8, wherein the formulation is used to coat a layered hydrophobic surface.

11. A sensor system for detecting a chemical comprising:
means for transmitting light;
means for sensing the transmitted light;
a formulation for coating the sensing means, the formulation comprising a dye forming chemistry, the dye forming chemistry reacting with the chemical to form a dye, the dye being opaque and thereby reducing the light sensed by the sensing means causing a decrease in voltage of the sensing means that is proportional to the concentration of the chemical, the voltage decrease being used to detect the presence of the chemical.

12. The sensor system as recited in claim 11, further comprising:
means for transmitting the voltage decrease; and
means for receiving the transmitted voltage decrease.

13. The sensor system as recited in claim 12, wherein the receiving means is remote from the voltage decrease transmitting means.

14. The sensor system as recited in claim 11, further comprising means for triggering an alarm when the concentration of the chemical reaches a threshold level.

15. The sensor system as recited in claim 12, further comprising means for deploying the light transmitting means, the coated sensing means and the voltage decrease transmitting means remotely from the receiving means.

16. The sensor system as recited in claim 11, wherein the formulation further comprises a solution for containing the dye forming chemistry, the solution comprising one of dichloromethane, toluene and chloroform and the chemical comprises phosgene.

17. The sensor system as recited in claim 16, the dye forming chemistry comprising 4-Nitrobenzylpyridine and benzylaniline.

18. The sensor system as recited in claim 17, the dye forming chemistry further comprising polystyrene.

19. The sensor system as recited in claim 17, wherein the formulation is used to coat a layered hydrophobic surface.

20. The sensor system as recited in claim 11, the light transmitting means comprising an LED and the sensing means comprising a phototransistor.
